(19) United States
(12) Patent Application Publication
(10) Pub. No.: US 2024/0325363 A1
(43) Pub. Date: Oct. 3, 2024

(54) COMPOSITION FOR TREATMENT OF ANTICANCER AGENT-RESISTANT CANCER

(71) Applicants: ONCOCROSS CO.,LTD., Seoul (KR); UNIVERSITY-INDUSTRY COOPERATION GROUP OF KYUNG HEE UNIVERSITY, Yongin-si, Gyeonggi-do (KR)

(72) Inventors: Yi Rang KIM, Sejong (KR); Sang Yun PARK, Seoul (KR); Jin Woo CHOI, Seongnam-si (KR); Soo Min LEE, Seoul (KR)

(73) Assignees: ONCOCROSS CO.,LTD., Seoul (KR); UNIVERSITY-INDUSTRY COOPERATION GROUP OF KYUNG HEE UNIVERSITY, Yongin-si, Gyeonggi-do (KR)

(21) Appl. No.: 18/281,480

(22) PCT Filed: Mar. 3, 2022
(86) PCT No.: PCT/KR2022/003003

§ 371 Date: Sep. 11, 2023

(30) Foreign Application Priority Data
Mar. 12, 2021 (KR) 10-2021-0032693

(51) Int. Cl. A61K 31/4439; A23L 33/10; A61P 35/00

(52) U.S. Cl. CPC A61K 31/4439; A23L 33/10; A61P 35/00

(57) ABSTRACT

The present invention relates to an anticancer agent capable of overcoming EGFR-TKI resistance in KRAS mutant cancer. GW8510 according to the present invention is an effective inhibitor against oncogenic mutation, especially, KRAS mutation, which is associated to resistance of the anticancer agent EGFR inhibitor and remarkably inhibits expression of both the wild-type and various mutants of KRAS, whereby the drug itself exhibits an anticancer effect, surmounts anticancer agent resistance, and noticeably promotes the death of anticancer agent-resistant cancer cells when used in combination with an anticancer agent. Thus, the inhibitor finds advantages applications in treating cancers, especially anticancer agent resistant cancers.

### TECHNICAL FIELD

The present invention relates to a composition for the treatment of anticancer agent-resistant cancer, and to an anticancer agent capable of overcoming EGFR-TKI resistance in KRAS mutant cancer.

### BACKGROUND ART

The smallest unit that constitutes the human body is called a cell, and normal cells divide, grow, and die and maintain cell number balance through intracellular regulatory functions. If a cell is damaged for some reason, the damaged cell is treated to serve as a normal cell, or dies by itself if the cell is not restored. However, when changes occur in the genes of a cell for various reasons, the cell changes abnormally, matures incompletely, and the cell cycle is not regulated and cell division continues, which is defined as cancer. These cancers affect all tissues and organs of the body with various degrees of prevalence. Many kinds of therapeutic agents for cancer has been developed over the last few decades to treat various types of cancer. The most commonly used types of anticancer agents include DNA-alkylating agents (e.g., cyclophosphamide, ifosfamide), antimetabolites (e.g., methotrexate, folic acid antagonists, and 5-fluorouracil, pyrimidine antagonists), microtubule disintegrants (e.g., vincristine, vinblastine, paclitaxel), DNA intercalators (e.g., doxorubicin, daunomycin, cisplatin), and hormonal therapies (e.g., tamoxifen, flutamide), etc.

Among them, EGFR inhibitors, which are targeted therapeutic agents, are largely divided into monoclonal antibodies and small molecule EGFR tyrosine kinase inhibitors. The epidermal growth factor receptor (EGFR), one of the HER family of tyrosine kinase receptors, mediates cell proliferation, angiogenesis, invasion, and metastasis (Harari P M, et al. J Clin Oncol. 2007; 25:4057-65; Yarden Y, et al. Nat Rev Mol Cell Biol. 2001; 2:127-37). The abnormal expression of EGFR is frequently observed in many tumors and is known to have strong oncogenic potential (Hirsch F R, et al. J Clin Oncol. 2003; 21:3798-807; Rubin Grandis J, et al. J Natl Cancer Inst. 1998; 90:824-32). First-generation EGFR tyrosine kinase inhibitors (TKIs), such as gefitinib and erlotinib, reversibly bind to ATP clefts within an EGFR kinase domain to inhibit autophosphorylation of EGFR (Mendelsohn J, et al. J Clin Oncol. 2003; 21:2787-99).

Meanwhile, KRAS (K-Ras), which is a Kirsten ras oncogene homolog of the mammalian ras gene family, is associated with cancer progression, clinical prognosis, and treatment outcomes, and KRAS mutations underlie the pathogenesis of about 20% of human cancers and occur in at least 90% of pancreatic cancer, at least 40% of colorectal cancer, and at least 30% of lung adenocarcinoma (Cox A D, et al. Nature Reviews. Drug Discovery. 13(11): 828-51). Mutant KRAS patients have a shorter survival time compared to other mutations, and it has been reported that KRAS mutation induces excessive activation of signaling pathways such as RAF-MEK-ERK and PI3K-AKT-mTOR to play an important role in cell growth, malignant transformation, and drug resistance. When KRAS is mutated, KRAS associated signals are activated regardless of EGFR activation, and thus there is no effect even in treating anti-EGFR inhibitors. The same result is shown even in patient data, and in the case of wild-type KRAS colorectal cancer, the survival rate is better when anti-EGFR inhibitors are administered than when only general conservative treatment is administered. However, in the case of KRAS mutant colon cancer, there is no effect even when administered with the anti-EGFR inhibitors. Based on these data, the use of the anti-EGFR inhibitors is currently excluded even in standard treatment in the case of KRAS mutant colorectal cancer. In particular, it has been reported that the KRAS mutations have great resistance to therapies of panitumumab (Vectibix) and cetuximab (Erbitux) in colon cancer, and have great resistance even to treatment with erlotinib (Tarceva) and gefitinib (Iressa) (Suda K, et al. Cancer Metastasis Reviews. 29 (1): 49-60). As such, although an RAS protein was the most frequently mutated gene in cancer, multi-targeted therapies attacking RAS may cause failure due to signaling, feedback loops, redundancy, tumor heterogeneity, and the absence of hydrophobic pockets capable of binding to small molecules on the RAS surface, and thus the RAS protein has been considered “undruggable”. For example, the development of AKT inhibitors has encountered extensive limitations, including high resistance, severe hyperglycemia, and other metabolic abnormalities, and the use of ERK inhibitors is limited by rash, diarrhea, peripheral edema, fatigue, and acne dermatitis as well as cardiac and ophthalmic side effects. Therefore, new targeted drug treatments capable of inhibiting these carcinogenic pathways are attracting a lot of research attention from scientists.

### DISCLOSURE

### Technical Problem

An object of the present invention is to provide a pharmaceutical composition for preventing or treating cancer.

Another object of the present invention is to provide an anticancer adjuvant.

Yet another object of the present invention is to provide a food composition for preventing or improving cancer.

Yet another object of the present invention is to provide a method for treating cancer.

Yet another object of the present invention is to provide a use for use in the preparation of a pharmaceutical composition for preventing or treating cancer.

### Technical Solution

One aspect of the present invention provides a pharmaceutical composition for preventing or treating cancer including GW8510 or a pharmaceutically acceptable salt thereof as an active ingredient.

Another aspect of the present invention provides an anticancer adjuvant including GW8510 or a pharmaceutically acceptable salt thereof as an active ingredient.

Yet another aspect of the present invention provides a food composition for preventing or improving cancer including GW8510 or a pharmaceutically acceptable salt thereof.

Yet another aspect of the present invention provides a method for treating cancer including administering GW8510 or a pharmaceutically acceptable salt thereof in a pharmaceutically effective dose to a subject suffering from cancer.

Yet another aspect of the present invention provides a use of GW8510 or a pharmaceutically acceptable salt thereof for use in the preparation of a pharmaceutical composition for preventing or treating cancer.

### Advantageous Effects

According to the present invention, GW8510 is an effective inhibitor against oncogenic mutation, especially, KRAS mutation, which is associated to resistance of the anticancer agent EGFR inhibitor and remarkably inhibits expression of both the wild-type and various mutants of KRAS, whereby the drug itself exhibits an anticancer effect, surmounts anticancer agent resistance, and noticeably promotes the death of anticancer-resistant cancer cells when used in combination with an anticancer agent. Thus, the inhibitor finds advantages applications in treating cancers, especially anticancer agent resistant cancers.

### DESCRIPTION OF DRAWINGS

FIG. 1 is a diagram illustrating screening of drugs capable of inhibiting KRAS (K-Ras) by performing bioinformatics analysis:
A: DEG Heatmap of CRC patients;
B: Schematic diagram of drug-induced gene network changes in a KRAS expression 20% high group and a KRAS expression 20% low group;
C: Schematic diagram of genes reconstructed by drugs; and
D: Chemical structure of GW8510.

FIG. 2 is a diagram confirming a KRAS expression inhibitory effect of GW8510 in a cancer cell line HCT116 with a KRAS G13D mutation.

FIG. 3 is a diagram confirming expression inhibitory effects of GW8510 on KRAS, XIAP and NFYA in a cancer cell line HCT116 with a KRAS G13D mutation.

FIG. 4 is a diagram confirming expression inhibitory effects of GW8510 on KRAS, XIAP and NFYA by Endpoint PCR in cancer cell lines with various KRAS mutations.

FIG. 5 is a diagram confirming decreases in KRAS expression levels by GW8510 in cancer cells with various KRAS mutations by Endpoint PCR (A), qRT-PCR (B), phenotype (C), and immunoblot (D).

FIG. 6 is a diagram confirming anticancer activity of GW8510 and anticancer activities by combined treatment with EGFR inhibitor anticancer agents (cetuximab as an anti-EGFR monoclonal antibody, and erlotinib as an anti-EGFR tyrosine receptor kinase inhibitor) in an HCT116 cell line as anticancer agent-resistant cancer.

FIG. 7 is a diagram confirming in vivo the anticancer activity of GW8510 and/or the anticancer activity of an EGFR inhibitor anticancer agent (cetuximab as an anti-EGFR monoclonal antibody).

FIG. 8 is a diagram illustrating results of cell cycle analysis and comparison of the KRAS expression inhibitory effects of GW8510 and other Cdk inhibitors in an HCT116 cell line:
A: Endpoint PCR data for KRAS expression of GW8510 and other Cdk inhibitors;
B: qRT-PCR data for KRAS expression of GW8510 and other Cdk inhibitors;
C: PI staining flow cytometry data; and
D: Cell cycle change group change graph.

FIG. 9 is a diagram comparing KRAS expression inhibitory effects of GW8510 and other Cdk inhibitors in an HCT116 cell line by Endpoint PCR.

FIG. 10 is a schematic diagram illustrating a normal EGFR pathway (A), a mechanism of anticancer activity by an EGFR inhibitor (B), a mechanism of EGFR inhibitor resistant cancer caused by KRAS mutation (C), and a death mechanism of EGFR inhibitor resistant cancer through KRAS mutation inhibition of GW8510 (D).

### BEST MODE FOR THE INVENTION

Hereinafter, an embodiment of the present invention will be described in detail with reference to the accompanying drawings. However, the following embodiments are presented as examples for the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

Terminologies used herein are terminologies used to properly express preferred embodiments of the present invention, which may vary according to a user, an operator's intention, or customs in the art to which the present invention pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

Throughout the present specification, general one-letter or three-letter codes for naturally existing amino acids are used, and generally allowed three-letter codes for other amino acids, such as α-aminoisobutyric acid (Aib), and N-methylglycine (Sar) are also used. The amino acids mentioned herein as abbreviations are also described as follows according to the IUPAC-IUB nomenclature.

Alanine: A, Arginine: R, Asparagine: N, Aspartic acid: D, Cysteine: C, Glutamic acid: E, Glutamine: Q, Glycine: G, Histidine: H, Isoleucine: I, Leucine: L, Lysine: K, Methionine: M, Phenylalanine: F, Proline: P, Serine: S, Threonine: T, Tryptophan: W, Tyrosine: Y, and Valine: V.

In one aspect, the present invention relates to a pharmaceutical composition for preventing or treating cancer including GW8510 or a pharmaceutically acceptable salt thereof as an active ingredient.

In one embodiment, the GW8510 may be represented by Chemical Formula 1:

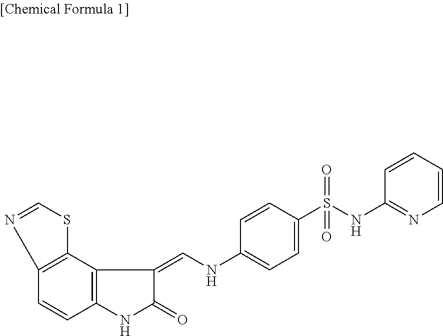 [SMILES: O=C1Nc2ccc3ncsc3c2/C1=C/Nc1ccc(S(=O)(=O)Nc2ccccn2)cc1]

In one embodiment, the cancer may be solid cancer, and may be anticancer agent-resistant cancer, EGFR inhibitor-resistant cancer, and EGFR tyrosine kinase inhibitors (TKIs)-resistant cancer.

In one embodiment, the EGFR inhibitor may be dacomitinib, osimertinib, cetuximab, Pyrotinib, Lcotinib, panitumumab, zalutumumab, Nimotuzumab, matuzumab, gefitinib, erlotinib, Lapatinib, neratinib, vandetanib, necitumumab, afatinib, AP26113, EGFR inhibitor (CAS No. 879127-07-8), EGFR/ErbB-2/ErbB-4 inhibitor (CAS No. 881001-19-0), EGFR/ErbB-2 inhibitor (CAS No. 179248-61-4), EGFR inhibitor II (BIBX 1382, CAS No. 196612-93-8), EGFR inhibitor III (CAS No. 733009-42-2), EGFR/ErbB-2/ErbB-4 inhibitor II (CAS No. 944341-54-2) or PKCβII/EGFR inhibitor (CAS No. 145915-60-2).

In one embodiment, the cancer may be cancer with a KRAS mutation, and the KRAS mutation may be G13D, G12C, or G12D.

In one embodiment, the cancer may be any one selected from the group consisting of brain tumor, melanoma, non-melanoma skin cancer, myeloma, oral cancer, salivary gland cancer, liver cancer, hepatocellular carcinoma, stomach cancer, colorectal cancer, colorectal adenocarcinoma, breast cancer, lung cancer, small cell lung cancer, non-small cell lung cancer, bone cancer, pancreatic cancer, skin cancer, head or neck cancer, head and neck squamous cell carcinoma, colon cancer, small intestine cancer, rectal cancer, glioblastoma, fallopian tube carcinoma, perianal cancer, cervical cancer, ovarian cancer, endometrial cancer, uterine squamous cell carcinoma, uterine adenocarcinoma, vaginal carcinoma, vulvar carcinoma, Hodgkin's disease, esophageal cancer, esophagogastric adenocarcinoma, esophagogastric cancer, esophageal squamous cell carcinoma, lymph node cancer, bladder cancer, bladder urothelial carcinoma, upper urinary tract urothelial carcinoma, gallbladder cancer, endocrine cancer, thyroid cancer, parathyroid cancer, adrenal cancer, soft tissue sarcoma, germ cell tumor, pheochromocytoma, thymic tumor, urethral cancer, penile cancer, prostate cancer, chronic or acute leukemia, lymphocytic lymphoma, renal or ureteral cancer, diffuse glioblastoma, renal cell carcinoma, renal pelvic carcinoma, central nervous system tumor, primary central nervous system lymphoma, spinal cord tumor, ampullary carcinoma, adrenocortical carcinoma, brainstem glioma, and pituitary adenoma, and more preferably colon cancer, lung cancer, pancreatic cancer, prostate cancer, or breast cancer.

In one embodiment, the composition may further include an XIAP or NYFA expression inhibitor.

As used herein, the term “anticancer agent-resistance (drug-resistance)” refers to a symptom that has extremely low sensitivity to anticancer agent therapy, and does not exhibit improved, palliated, alleviated, or treated symptoms of cancer by the therapy. The anticancer agent-resistance may have resistance to specific anticancer agent therapy of cancer from the beginning and does not exhibit resistance initially, but the properties of cancer cells may change due to long-term treatment, and thus the cancer cells may no longer be sensitive to the same therapeutic agent. However, in the present invention, the anticancer agent resistance is caused by KRAS mutation.

The pharmaceutical composition of the present invention may further include a known anticancer agent in addition to GW8510 as an active ingredient, and may be used in combination with other known treatments for the treatment of these diseases. Other treatments include chemotherapy, radiation therapy, hormone therapy, bone marrow transplantation, stem-cell replacement therapy, other biological therapies, immunotherapy, and the like, but are not limited thereto.

As used herein, the term “prevention” means all actions that inhibit or delay the occurrence, spread, and recurrence of cancer by administration of the pharmaceutical composition according to the present invention. The term “treatment” means all actions that improve or beneficially change the symptoms of cancer by administering the composition of the present invention. Those skilled in the art to which the present invention pertains will be able to determine the degree of improvement, enhancement and treatment by knowing the exact criteria of a disease for which the composition of the present invention is effective by referring to data presented by the Korean Academy of Medical Sciences, etc.

As used herein, the term “therapeutically effective dose” used in combination with the active ingredients means an amount effective to prevent or treat a target disease, and the therapeutically effective dose of the composition of the present invention may vary depending on several factors, such as a method of administration, a target site, the condition of a patient. Accordingly, when used in the human body, the dose should be determined as an appropriate amount in consideration of both safety and efficiency. It is also possible to estimate the amount used in humans from the effective dose determined through animal experiments. These matters to be considered when determining the effective dose are described in, for example, Hardman and Limbird, eds., Goodman and Gilman's The Pharmacological Basis of Therapeutics, 10th ed. (2001), Pergamon Press; and E.W. Martin ed., Remington's Pharmaceutical Sciences, 18th ed. (1990), Mack Publishing Co.

The pharmaceutical composition of the present invention is administered in a pharmaceutically effective dose. As used herein, the term ‘pharmaceutically effective dose’ refers to an amount enough to treat the disease at a reasonable benefit/risk ratio applicable to medical treatment and enough not to cause side effects. The effective dose level may be determined according to factors including the health condition of a patient, the type and severity of cancer, the activity of a drug, the sensitivity to a drug, a method of administration, a time of administration, a route of administration, an excretion rate, duration of treatment, and drugs used in combination or simultaneously, and other factors well-known in the medical field. The composition of the present invention may be administered as an individual therapeutic agent or in combination with other therapeutic agents, and may be administered sequentially or simultaneously with existing therapeutic agents, and may be administered single time or multiple times. It is important to administer an amount capable of obtaining a maximum effect with a minimal amount without side effects by considering all the factors, which may be easily determined by those skilled in the art.

The pharmaceutical composition of the present invention may include carriers, diluents, excipients, or a combination of two or more thereof, which are commonly used in biological agents. As used herein, the term “pharmaceutically acceptable” means that the composition exhibits non-toxic properties to cells or humans exposed to the composition. The carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be preferably prepared according to each disease or ingredient using as a suitable method in the art or a method disclosed in Remington's Pharmaceutical Science (Mack Publishing Company, Easton PA, 18th, 1990).

In one embodiment, the pharmaceutical composition may be one or more formulations selected from the group consisting of oral formulations, external preparations, suppositories, sterile injections and sprays, and more preferably oral or injective formulations.

As used herein, the term “administration” means providing a predetermined substance to a subject or patient by any suitable method, and the pharmaceutical composition may be administered parenterally (e.g., applied as an injectable formulation intravenously, subcutaneously, intraperitoneally or topically) or orally according to a desired method. The dose range may vary depending on the body weight, age, sex, and health condition of a patient, a diet, an administration time, an administration method, an excretion rate, and the severity of a disease. Liquid formulations for oral administration of the composition of the present invention correspond to suspensions, internal solutions, emulsions, syrups, etc., and may include various excipients, such as wetting agents, sweeteners, fragrances, preservatives, and the like in addition to water and liquid paraffin, which are commonly used simple diluents. Formulations for parenteral administration include sterilized aqueous solutions, non-aqueous solvents, suspensions, emulsions, lyophilized agents, suppositories, and the like. The pharmaceutical composition of the present invention may also be administered by any device capable of transferring an active substance to a target cell. Preferred methods of administration and formulations are intravenous injections, subcutaneous injections, intradermal injections, intramuscular injections, or drop injections. The injections may be prepared by using aqueous solvents such as a physiological saline solution and a ringer solution, and non-aqueous solvents such as vegetable oils, higher fatty acid esters (e.g., ethyl oleate), and alcohols (e.g., ethanol, benzyl alcohol, propylene glycol, or glycerin). The injections may include pharmaceutical carriers, such as a stabilizer for the prevention of degeneration (e.g., ascorbic acid, sodium hydrogen sulfite, sodium pyrosulfite, BHA, tocopherol, EDTA, etc.), an emulsifier, a buffer for pH control, and a preservative to inhibit microbial growth (e.g., phenyl mercury nitrate, thimerosal, benzalkonium chloride, phenol, cresol, benzyl alcohol, etc.).

As used herein, the term “subject” refers to all animals including monkeys, cows, horses, sheep, pigs, chickens, turkeys, quails, cats, dogs, mice, rats, rabbits or guinea pigs including humans who have developed or may develop the cancer, and the pharmaceutical composition of the present invention may be administered to a subject to effectively prevent or treat the diseases. The pharmaceutical composition of the present invention may be administered in combination with existing therapeutic agents.

The pharmaceutical composition of the present invention may further include pharmaceutically acceptable additives. At this time, the pharmaceutically acceptable additives may use starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc and the like. The pharmaceutically acceptable additive according to the present invention is preferably included in an amount of 0.1 part by weight to 90 parts by weight based on the composition, but is not limited thereto.

In one aspect, the present invention provides an anticancer adjuvant including GW8510 or a pharmaceutically acceptable salt thereof as an active ingredient.

In one embodiment, the anticancer adjuvant of the present invention may be administered in combination with an anticancer agent.

In one embodiment, the anticancer adjuvant of the present invention may be administered simultaneously, separately or sequentially with or from the anticancer agent.

In one embodiment, the anticancer adjuvant of the present invention may inhibit resistance to the anticancer agent.

In one embodiment, the anticancer agent may be an EGFR inhibitor or EGFR tyrosine kinase inhibitors (TKIs).

In one embodiment, the anticancer agent may be dacomitinib, osimertinib, cetuximab, Pyrotinib, Lcotinib, panitumumab, zalutumumab, Nimotuzumab, matuzumab, gefitinib, erlotinib, Lapatinib, neratinib, vandetanib, necitumumab, afatinib, AP26113, EGFR inhibitor (CAS No. 879127-07-8), EGFR/ErbB-2/ErbB-4 inhibitor (CAS No. 881001-19-0), EGFR/ErbB-2 inhibitor (CAS No. 179248-61-4), EGFR inhibitor II (BIBX 1382, CAS No. 196612-93-8), EGFR inhibitor III (CAS No. 733009-42-2), EGFR/ErbB-2/ErbB-4 inhibitor II (CAS No. 944341-54-2) or PKCβII/EGFR inhibitor (CAS No. 145915-60-2).

As used herein, the term “anticancer adjuvant” is a preparation that may improve, enhance or increase the anticancer effect of an anticancer agent, and does not show anticancer activity by itself, but may be a preparation capable of improving, enhancing or increasing the anticancer effect of the anticancer agent when used together with an anticancer agent. In addition, when a preparation exhibiting a concentration-dependent anticancer activity is used together with an anticancer agent at a level that does not exhibit anticancer activity by itself, the preparation may be a preparation capable of improving, enhancing or increasing the anticancer effect of the anticancer agent.

The route of administration of the anticancer adjuvant may be administered through any general route as long as the anticancer adjuvant may reach a target tissue. The anticancer adjuvant of the present invention may be administered intraperitoneally, intravenously, intramuscularly, subcutaneously, intradermally, orally, intranasally, pulmonaryly, or intrarectally according to a desired purpose, but is not limited thereto. In addition, the anticancer adjuvant may be administered by any device capable of transferring an active substance to a target cell.

In one aspect, the present invention relates to a food composition for preventing or improving cancer including GW8510 or a pharmaceutically acceptable salt thereof.

In one embodiment, the cancer may be EGFR inhibitor-resistant cancer and may be cancer with KRAS mutation.

When the composition of the present invention is used as the food composition, the GW8510 may be added as it is or used with other foods or food ingredients, and may be appropriately used according to a general method. The composition may include food supplement additives that are foodologically acceptable in addition to the active ingredients, and the mixing amount of the active ingredients may be appropriately determined depending on the purpose of use (prevention, health or therapeutic treatment).

As used herein, the term “food supplement additive” means a component that may be supplementally added to food, and may be appropriately selected and used by those skilled in the art as being added to prepare a health functional food of each formulation. Examples of the food supplement additives include various nutrients, vitamins, minerals (electrolytes), flavors such as synthetic and natural flavors, colorants and fillers, pectic acid and salts thereof, alginic acid and salts thereof, organic acids, protective colloidal thickeners, pH adjusters, stabilizers, preservatives, glycerin, alcohols, carbonation agents used in carbonated drinks, and the like, but the types of food supplement additives of the present invention are not limited by the examples.

The food composition of the present invention may include a health functional food. As used herein, the term “health functional food” refers to food prepared and processed in the form of tablets, capsules, powders, granules, liquids and pills by using raw materials or ingredients having functionalities useful to the human body. Here, the ‘functionality’ means regulating nutrients to the structure and function of the human body or obtaining effects useful for health applications such as physiological action. The health functional food of the present invention is able to be prepared by methods to be commonly used in the art and may be prepared by adding raw materials and ingredients which are commonly added in the art in preparation. In addition, the formulations of the health functional food may also be prepared with any formulation recognized as a health functional food without limitation. The food composition of the present invention may be prepared in various types of formulations, and unlike general drugs, the food composition has an advantage that there is no side effect that may occur when taking a long-term use of the drug by using the food as a raw material, and has excellent portability, so that the health functional food of the present invention can be taken as supplements to enhance the effects of anticancer agents.

In addition, there is no limitation in a type of health food in which the composition of the present invention may be used. In addition, the composition including the GW8510 of the present invention as the active ingredient may be prepared by mixing known additives with other suitable auxiliary ingredients that may be contained in the health functional food according to the selection of those skilled in the art. Examples of foods to be added include meat, sausage, bread, chocolate, candy, snacks, confectionery, pizza, ramen, other noodles, gum, dairy products including ice cream, various soups, beverages, tea, drinks, alcoholic beverages, vitamin complexes and the like, and may be prepared to be added to extract, tea, jelly, juice, and the like prepared by using the extract according to the present invention as a main ingredient.

In one aspect, the present invention relates to a method for treating cancer including administering GW8510 or a pharmaceutically acceptable salt thereof in a pharmaceutically effective dose to a subject suffering from cancer.

In one aspect, the present invention relates to a use of GW8510 or a pharmaceutically acceptable salt thereof for use in the preparation of a pharmaceutical composition for preventing or treating cancer.

### Modes for the Invention

Hereinafter, the present invention will be described in more detail with reference to the following Examples. However, the following Examples are only intended to embody the contents of the present invention, and the present invention is not limited thereto.

### Example 1. Screening of KRAS Targeted Drugs

Drugs capable of inhibiting KRAS (K-Ras) were screened by performing bioinformatics analysis. Specifically, RNA-seq data of colorectal cancer (CRC) was downloaded from a Cancer Genome Atlas Program (TCGA) with respect to expression levels of differentially expressed genes (DEGs) in a total of 331 samples (57 patients in a KRAS high group and 56 patients in a KRAS low group), and statistically processed using an AI platform to screen gene candidates with significant expression between a control group and a comparative group. Additionally, DEGs of all drugs were extracted from a Connectivity MAP (CMAP) database. CRC patients were divided into two groups according to KRAS expression (a KRAS expression 20% high group and a KRAS expression 20% low group) (FIG. 1A), and the gene expression patterns of the two groups were compared to obtain differentially expressed genes (DEGs) (FIG. 1B). As a result of determining a drug candidate capable of changing a gene pattern from the KRAS expression 20% high group to the KRAS expression 20% low group by analyzing the CRC DEGs and drug DEGs (FIG. 1C), GW8510, a cyclin-dependent kinase 2 inhibitor of Chemical Formula 1 below, was selected as a KRAS expression inhibitor (FIG. 1D).

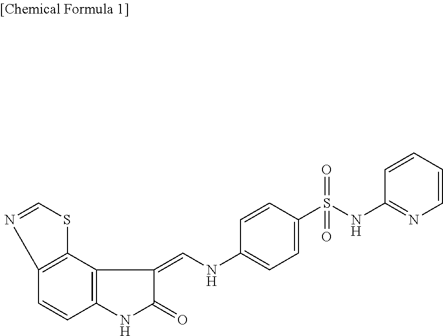 [SMILES: O=C1Nc2ccc3ncsc3c2/C1=C/Nc1ccc(S(=O)(=O)Nc2ccccn2)cc1]

### Example 2. Confirmation of Effect of GW8510 in Cancer Cells with Various KRAS Mutations

2-1. Confirmation of Effect of Inhibiting KRAS mRNA Expression in Cancer Cells with KRAS Mutations

In order to confirm the effect of inhibiting KRAS expression by GW8510, a human colon cancer cell line HCT116 with a KRAS G13D mutation, a human prostate cancer cell line PC-3 with a KRAS G13D mutation, a human breast cancer cell line MCF7 as a KRAS wild-type cell line, a human pancreatic cancer cell line MIA PaCa-2 with a KRAS G12C mutation, and a human pancreatic cancer cell line PANC-1 with a KRAS G12D mutation were dispensed in a 6-well plate, and treated with GW8510 at 0, 1, 5 or 10 μM of concentration for 3, 6, 24 or 48 hours. Thereafter, RNA was extracted from the cells using a TRIzol™ Reagent (Thermo Fisher Scientific Inc., USA) and then cDNA was synthesized using M-MLV reverse-transcriptase (ELPIS Biotech, Korea), 2.5 mM dNTP (TransGen Biotech, China), and oligo dT15 (bioneer, Korea). Endpoint PCR was performed using an Endpoint PCR solution mixed with the synthesized cDNA, Taq polymerase master mix, and primers shown in Table 1 below, and a QuantStudio 3 Real-Time PCR Instrument (Thermo Fisher Scientific Inc., USA). Thereafter, the mRNA level of KRAS was confirmed by loading the Endpoint PCR solution on an agarose gel. In addition, the mRNA levels of nuclear transcription factor Y (NYFA) and X-linked inhibitor of apoptosis protein (XIAP) as a protein of inhibiting apoptosis, were also confirmed together with KRAS.

TABLE 1

| | Classifi- | |
|---|---|---|
| Gene | cation | Sequence |
| KRAS | Forward | 5′-GAGGCCTGCTGAAAATGACTG-3′ |
| | Reverse | 5′-ATTACTACTTGCTTCCTGTAGG-3′ |
| GAPDH | Forward | 5′-CAAAAGGGTCATCATCTCTG-3′ |
| | Reverse | 5′-CCTGCTTCACCACCTTCTTG-3′ |

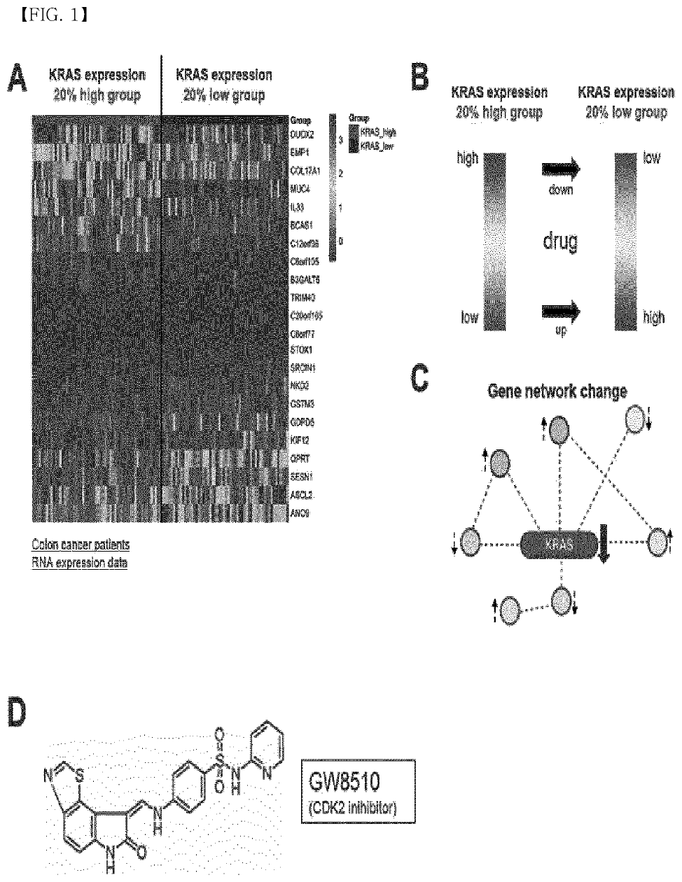

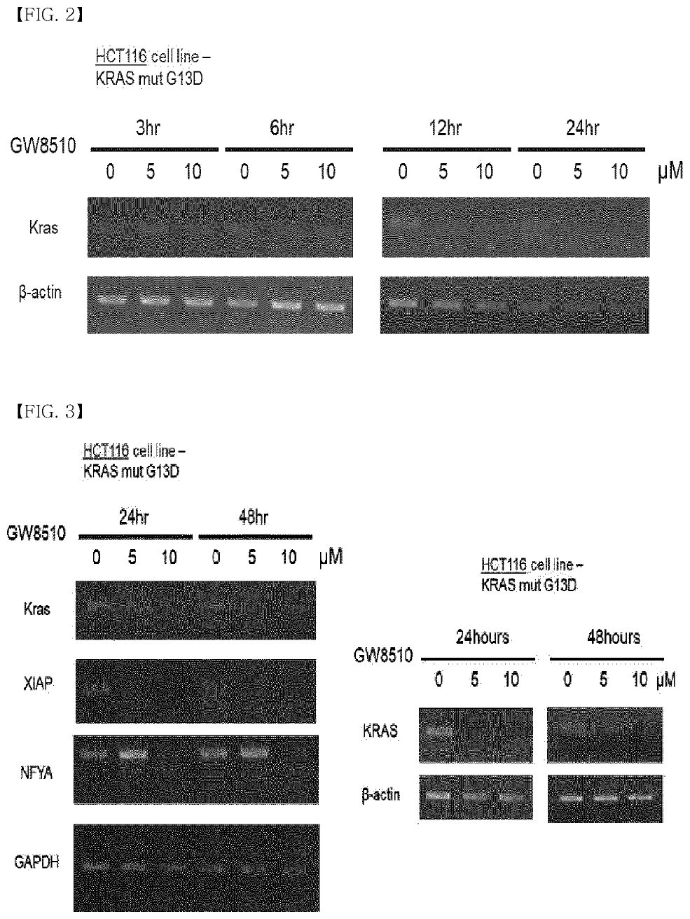

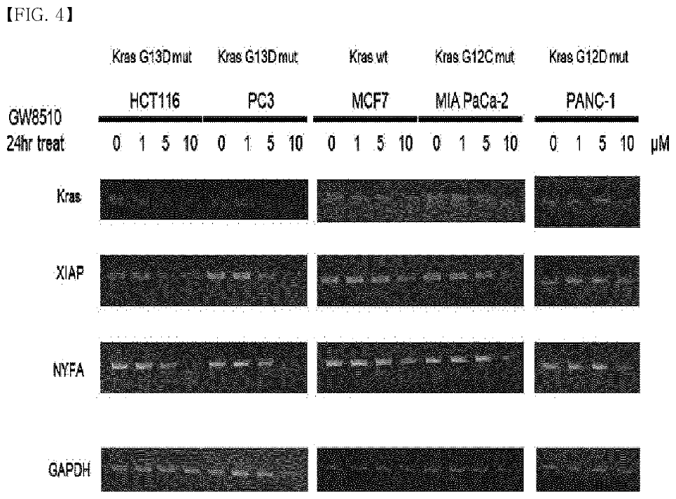

As a result, in colon cancer cells with KRAS G13D mutation, it was shown that the mRNA expression level of KRAS was decreased in a concentration-dependent manner of GW8510 from 6 hours after GW8510 treatment (FIG. 2), and the mRNA expression levels of XIAP and NFYA were also decreased (FIG. 3). In addition, it was confirmed that in all of the human prostate cancer cell line PC-3 with the KRAS G13D mutation, the human pancreatic cancer cell line MIA PaCa-2 with the KRAS G12C mutation, and the human pancreatic cancer cell line PANC-1 with the KRAS G12D mutation, the KRAS mRNA levels were decreased by treatment of GW8510. It was confirmed that the KRAS mRNA level was also reduced in the human breast cancer cell line MCF7 as a KRAS wild-type cell line, and thus, the KRAS transcription level was significantly reduced by GW8510 in all of these cells. In addition, it was shown that the mRNA levels of XIAP and NFYA were also decreased in all of the cell lines (FIG. 4).

2-2. Confirmation of Effect of Inhibiting KRAS Protein Expression in Cancer Cells with KRAS Mutations

A colon cancer cell line HCT116 (KRAS G13D mut), pancreatic cancer cell lines PANC-1 (KRAS G12D mut) and MIA PaCa-2 (KRAS G12C mut), and a breast cancer cell line MCF7 (KRAS wt) were dispensed in a 6-well plate, and treated with GW8510 at 0, 1, 5 or 10 μM of concentration for 24 hours, and then Endpoint PCR was performed. In addition, a qRT-PCR solution was prepared by mixing SYBR Green Master Mix, the cDNA of Example 2-1, and the primers in Table 1, and qRT-PCR was performed with QuantStudio 3 Real-Time PCR Instrument (Thermo Fisher Scientific Inc., USA) to quantify the gene expression levels. In addition, the phenotypes of the cancer cell line were observed by treatment of GW8510 by using the colon cancer cell line HCT116 treated with GW8510 at 0 or 10 μM of concentration for 24 hours, and the protein expression levels of pan ras, K-ras, and β-actin were measured by immunoblot. Specifically, the colon cancer cell line HCT116 treated with GW8510 at 0 or 10 μM of concentration for 24 hours was lysed with an RIPA buffer (Curebio, Korea), and then the cell lysate was centrifuged and the supernatant was taken. The cell lysate supernatant was added with a 5×SDS sample buffer and then boiled for 10 minutes, and electrophoresis was performed using a 12% SDS polyacrylamide gel. After electrophoresis, a protein was transferred to a PVDF membrane and then blocked with 5% skim milk (TBS-T) at room temperature for 1 hour. Thereafter, primary antibodies (anti-pan ras, anti-K-ras, and anti-β-actin) diluted in a blocking solution at 1:1000 were added and incubated at 4° C. overnight, and then a membrane was washed three times with 0.05% TBS-T, and a secondary antibody (horseradish peroxidase-conjugated 2nd Ab) diluted at 1:10000 was added and incubated for 1 hour at room temperature. The membrane was washed three times with 0.05% TBS-T, developed with an enhanced chemiluminescence (ECL) solution, and exposed to an X-ray film.

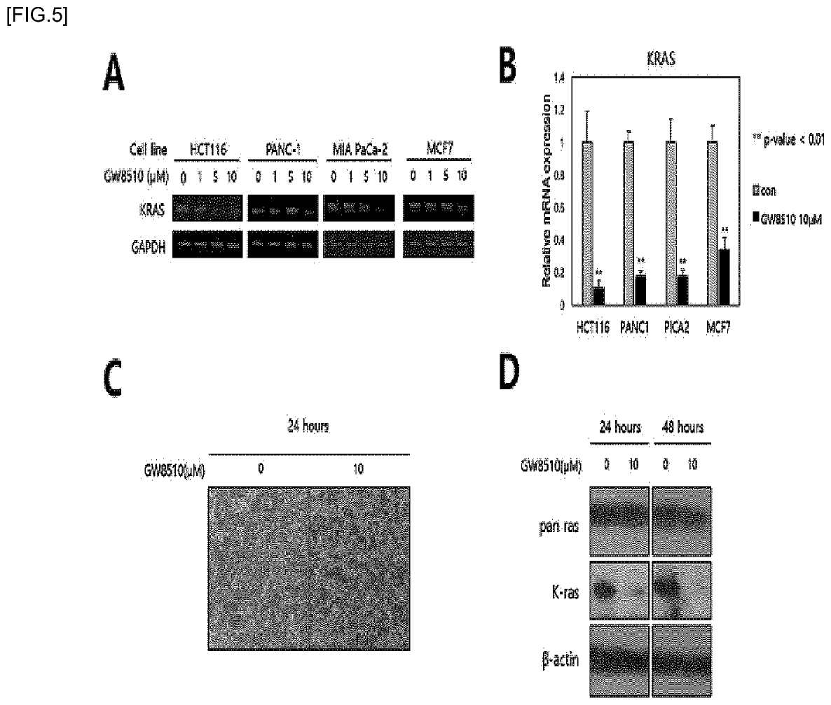

As a result, as in Example 2-1, it was confirmed that the KRAS transcription level was significantly reduced by GW8510 in all these cells (FIGS. 5A and 5B), and the KRAS protein level was also decreased (FIG. 5D).

Through this, it was confirmed that GW8510 reduced the KRAS expression regardless of the mutation and type of KRAS.

### Example 3. Confirmation of Cancer Cell Killing Effect of GW8510 and Combination Effect with Anticancer Agent

Due to KRAS gene mutation, which was the most common oncogenic mutation that occurred in metastatic cancer, GW8510 had resistance to an EGFR inhibitor used as an anticancer agent. As a result, in order to confirm an anticancer effect of GW8510, which transcriptionally inhibited the expressions of KRAS and KRAS mutation and to confirm the anticancer effect by combined treatment of GW8510 and the anticancer agent, the HCT116 cell line was treated with 10 μM GW8510 and 25 μM the EGFR inhibitor cetuximab or erlotinib for 24 or 48 hours, and then the survival of the cancer cell line was confirmed through MTT analysis. Specifically, the HCT116 cells were dispensed in a 96-well plate, and one day later, treated with 10 μM GW8510 and 25 μM cetuximab or erlotinib. Thereafter, the cells were treated with 10 μl of an MTT solution (5 mg/ml) and 90 μl of an RPMI medium and incubated in a 5% CO₂ incubator for 3 to 4 hours. The medium was removed, and formazan crystals were dissolved in 100 μl of DMSO. Absorbance was measured at 570 nm in a microplate reader, and cell viability was quantified by an OD measured value of the dissolved formazan crystals.

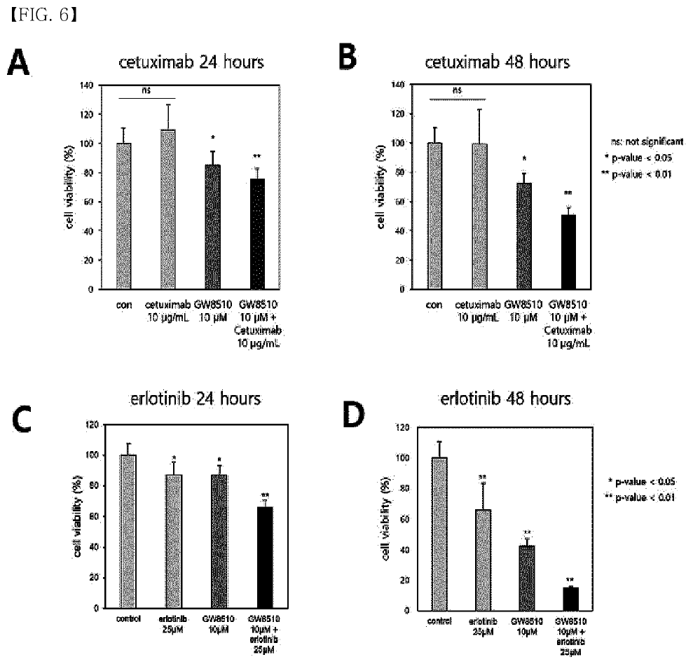

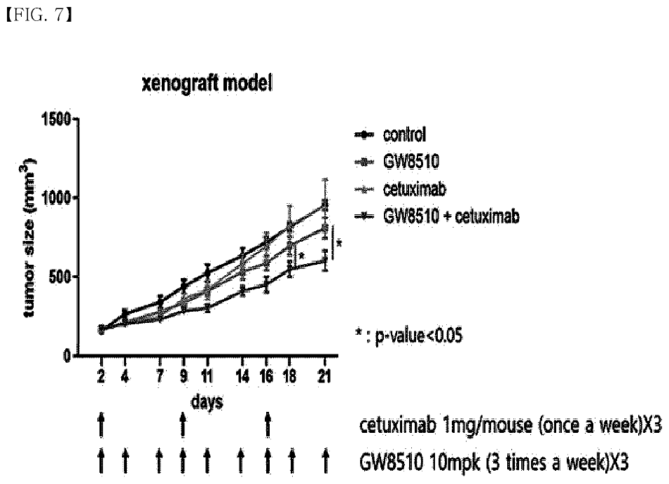

As a result, there was little cytotoxicity when treated with cetuximab, but the anticancer activity was shown when GW8510 was administered alone, and particularly, it was shown that when the two drugs were treated in combination, the cancer cell killing effect was significantly increased (FIGS. 6A and 6B). In addition, when erlotinib and GW8510 were treated in combination, the cell death of cancer cell lines with KRAS mutations was significantly increased synergistically (FIGS. 6C and 6D).

### Example 4. Confirmation of In Vivo Anticancer Effect of GW8510 and Combination Effect with Anticancer Agent

To confirm whether GW8510 had a cancer (tumor) inhibitory effect not only in vitro but also in vivo, a xenograft model was constructed using HCT116 cells in nude mice. Experimental animals used were 36 6-week-old Balb/c nude mice. The experimental diet and drinking water were taken freely, and all experimental diets were stored in a refrigerator during a breeding period. The mice were pre-bred with regular feed to be adapted to a breeding environment for 7 days before the start of the experiment and then divided into a total of 4 groups. The 4 groups were divided into a control group (n=9), a gw8510 experimental group (n=9), a cetuximab experimental group (n=9), and a gw8510 and cetuximab combined experimental group (n=9). In all mice, HCT116 cells were injected with 1×10⁶ S.C. at 7 weeks of age to prepare a xenograft model. When the tumor size reached 150 mm³ in the prepared tumor transplant model, the mice were divided into GW8510 alone, cetuximab alone, and GW8510 and cetuximab combined treatment groups, respectively, and GW8510 was administered intraperitoneally at a concentration of 10 mg/kg three times a week, and cetuximab was administered intraperitoneally at a concentration of 1 mg/mouse once a week for a total of three times. The tumor size was measured using Equation of (long axis×short axis×short axis)/2 and measured three times a week at the same time.

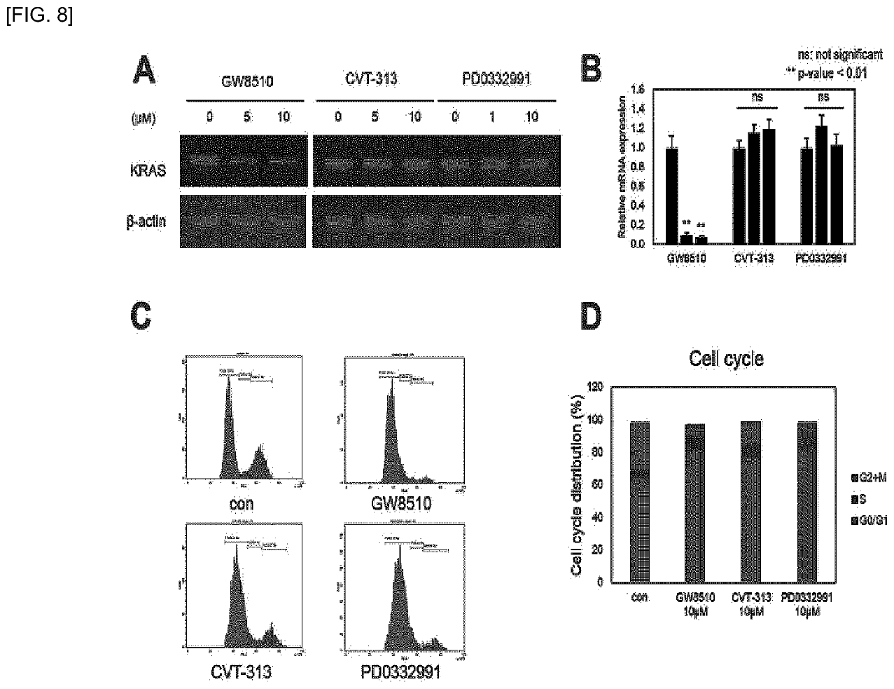

As a result, the tumor size that was not reduced by cetuximab administration tended to decrease compared to the control group when GW8510 was administered, and particularly, in the group administered with GW8510 and cetuximab in combination, it was shown that the tumor size was synergistically reduced (FIG. 7).

Through this, cancer with KRAS mutation had resistance to an EGFR inhibitor used as an anticancer agent, especially cetuximab to have no anticancer effect thereon, but it was confirmed even in vivo that GW8510, which transcriptionally inhibited the expression of KRAS mutation, had an anticancer effect alone, and increased sensitivity to have a synergistic anticancer effect when treated with an EGFR inhibitor in combination.

### Example 5. Confirmation of KRAS Changes by Treatment with Other CDK Inhibitors

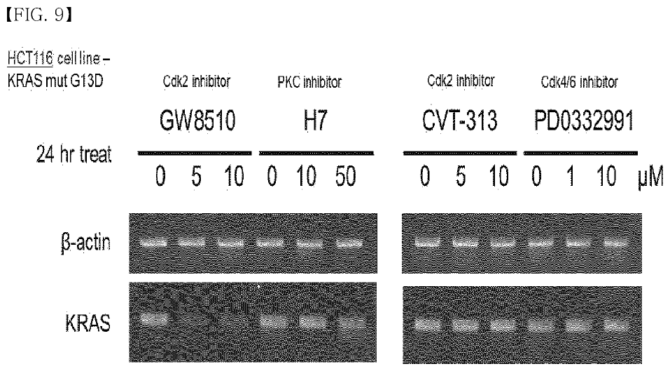

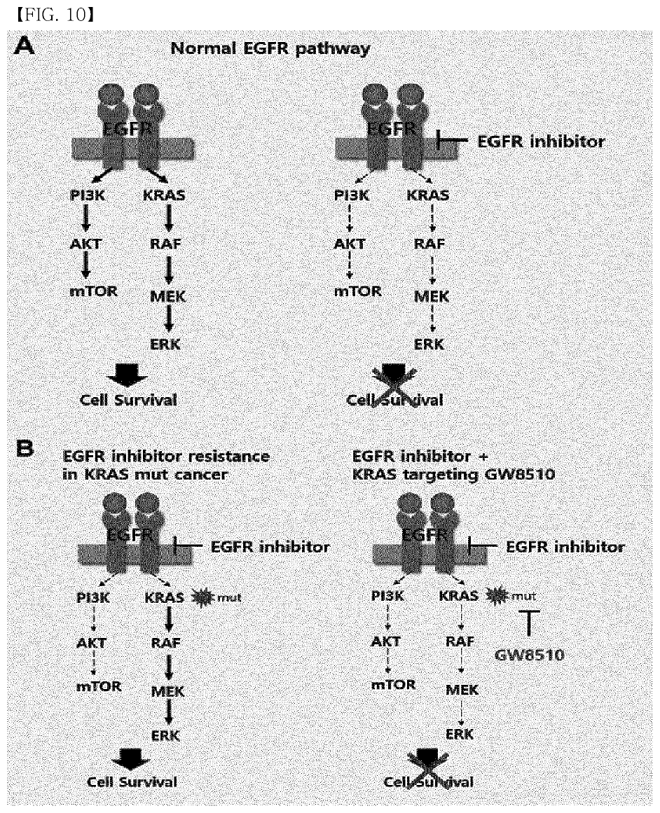

To determine whether the inhibition of KRAS by GW8510 was regulated through Cdk2 inhibition, HCT116 cells were treated with other CDK inhibitors, such as PKC inhibitor H7, Cdk2 inhibitor CVT-313, and Cdk4/6 inhibitor PD0332991, other than GW8510 (Cdk2 inhibitor) for 24 hours, respectively, and then to analyze the cell cycle, the cells were subjected to PI staining and confirmed by flow cytometry, and the KRAS expression levels were confirmed by Endpoint PCR and qRT-PCR. As a result, it was confirmed that the cell cycle was arrested in the GO/G1 phase and that CDK was well inhibited (FIGS. 8C and 8D), but there was no change in KRAS expression (FIGS. 8A and 8B), and only the GW8510 of the present invention inhibited the KRAS expression (FIG. 9).

1. A method for treating cancer comprising administering a pharmaceutical composition comprising GW8510 or a pharmaceutically acceptable salt thereof as an active ingredient to a subject in need thereof.

2. The method of claim 1, wherein the GW8510 is represented by Chemical Formula 1 below:

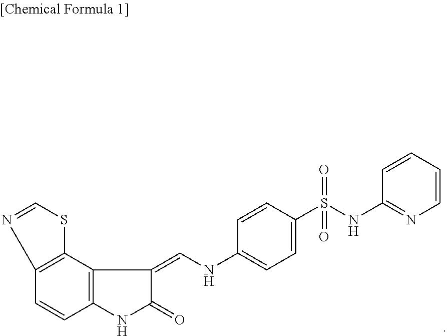 [SMILES: O=C1Nc2ccc3ncsc3c2/C1=C/Nc1ccc(S(=O)(=O)Nc2ccccn2)cc1]

3. The method of claim 1, wherein the cancer is solid cancer.

4. The method of claim 1, wherein the cancer is anticancer agent-resistant cancer.

5. The method of claim 1, wherein the cancer is EGFR inhibitor-resistant cancer.

6. The method of claim 5, wherein the EGFR inhibitor is dacomitinib, osimertinib, cetuximab, Pyrotinib, Lcotinib, panitumumab, zalutumumab, Nimotuzumab, matuzumab, gefitinib, erlotinib, Lapatinib, neratinib, vandetanib, necitumumab, afatinib, AP26113, EGFR inhibitor (CAS No. 879127-07-8), EGFR/ErbB-2/ErbB-4 inhibitor (CAS No. 881001-19-0), EGFR/ErbB-2 inhibitor (CAS No. 179248-61-4), EGFR inhibitor II (BIBX 1382, CAS No. 196612-93-8), EGFR inhibitor III (CAS No. 733009-42-2), EGFR/ErbB-2/ErbB-4 inhibitor II (CAS No. 944341-54-2) or PKCβII/EGFR inhibitor (CAS No. 145915-60-2).

7. The method of claim 1, wherein the cancer is EGFR tyrosine kinase inhibitors (TKIs)-resistant cancer.

8. The method of claim 1, wherein the cancer is cancer with KRAS mutation.

9. The method of claim 8, wherein the KRAS mutation is G13D, G12C or G12D.

10. An anticancer adjuvant comprising GW8510 or a pharmaceutically acceptable salt thereof as an active ingredient.

11. The anticancer adjuvant of claim 10, wherein the anticancer adjuvant is administered with an anticancer agent in combination.

12. The anticancer adjuvant of claim 11, wherein the anticancer adjuvant is administered with an anticancer agent simultaneously, separately, or sequentially.

13. The anticancer adjuvant of claim 11, wherein the anticancer agent is an EGFR inhibitor.

14. The anticancer adjuvant of claim 11, wherein the anticancer agent is EGFR tyrosine kinase inhibitors (TKIs).

15. The anticancer adjuvant of claim 11, wherein the anticancer agent is dacomitinib, osimertinib, cetuximab, Pyrotinib, Lcotinib, panitumumab, zalutumumab, Nimotuzumab, matuzumab, gefitinib, erlotinib, Lapatinib, neratinib, vandetanib, necitumumab, afatinib, AP26113, EGFR inhibitor (CAS No. 879127-07-8), EGFR/ErbB-2/ErbB-4 inhibitor (CAS No. 881001-19-0), EGFR/ErbB-2 inhibitor (CAS No. 179248-61-4), EGFR inhibitor II (BIBX 1382, CAS No. 196612-93-8), EGFR inhibitor III (CAS No. 733009-42-2), EGFR/ErbB-2/ErbB-4 inhibitor II (CAS No. 944341-54-2) or PKCβII/EGFR inhibitor (CAS No. 145915-60-2).

16. The anticancer adjuvant of claim 10, wherein the anticancer adjuvant inhibits resistance to the anticancer agent.

17. A method for improving cancer comprising administering a food composition comprising GW8510 or a pharmaceutically acceptable salt thereof.

18. The method of claim 17, wherein the cancer is EGFR inhibitor-resistant cancer.

19. The method of claim 17, wherein the cancer is cancer with KRAS mutation.

20. (canceled)

21. (canceled)
